(19) United States
(12) Patent Application Publication
(10) Pub. No.: US 2019/0336075 A1
(43) Pub. Date: Nov. 7, 2019

(54) DEVICE FOR MEASURING BIOLOGICAL INFORMATION

(71) Applicants: ALPS ALPINE CO., LTD., Tokyo (JP); Genial Light CO., LTD., Shizuoka (JP)

(72) Inventors: Toshio KAWANO, Miyagi (JP); Yukio OTAKI, Miyagi (JP); Ryo SHIMOKITA, Shizuoka (JP)

(21) Appl. No.: 16/514,317

(22) Filed: Jul. 17, 2019

Related U.S. Application Data
(63) Continuation of application No. PCT/JP2018/009274, filed on Mar. 9, 2018.

(30) Foreign Application Priority Data
Mar. 30, 2017 (JP) 2017-066903

(51) Int. Cl. A61B 5/00; A61B 5/04

(52) U.S. Cl. CPC A61B 5/684; A61B 5/04; A61B 5/0075

(57) ABSTRACT

A device for measuring biological information includes at least one light emitting element configured to emit light having predetermined wavelength, a light receiving element configured to receive returned light from a subject with respect to the emitted light, an attachment determination unit configured to determine whether the device is attached to the subject with respect to the light receiving element based on an amount of returned light received, and a measurement-point determination unit configured to determine whether or not a point where the device is attached to the subject is suitable for measuring the biological information based on the amount of returned light received, when the attachment determination unit determines that the device is attached to the subject.

### CROSS-REFERENCE TO RELATED APPLICATION

This application is a continuation application of International Application No. PCT/JP2018/009274 filed on Mar. 9, 2018, and designated the U.S., which is based upon and claims priority to Japanese Patent Application No. 2017-066903, filed on Mar. 30, 2017, the entire contents of which are incorporated herein by reference.

### BACKGROUND OF THE INVENTION

### 1. Field of the Invention

The present disclosure relates to a device for measuring biological information, and more particularly, to measuring biological information about a human body as a subject.

### 2. Description of the Related Art

A biological-information measuring device disclosed in Japanese Unexamined Patent Application Publication No. 2016-198193 (Patent Document 1) is attached to a user's body, and measures biological information related to the user. The biological-information measuring device includes a pulse-wave detecting unit for detecting a user's pulse wave to output a pulse signal, and a body-motion detecting unit for detecting a motion of a user's body to output a body motion signal. The biological-information measuring device further includes a state evaluating unit for evaluating, based on the body motion signal, a stability level from a state where the user exercises, and a detection-interval setting unit for setting a detection interval between pulse waves based on an evaluation result obtained by the state evaluating unit. In such a device, when a stability level from the state where the user exercises is evaluated to be sufficiently high, it is possible to change a setting so as to increase a detection interval between the pulse signals, thereby reducing power consumption.

However, with respect to a human body used as a subject, since distances from the skin to blood vessels in the skin vary according to a region of the body, even if measurement is performed using the biological-information measuring device disclosed in Patent Document 1, signals obtained vary in intensity, depending on a point where such a device is attached to the body. For this reason, if the signals are decreased, accurate measurement may not be performed.

### SUMMARY OF THE INVENTION

One or more embodiments in the present disclosure provide a device for measuring biological information. The device includes at least one light emitting element configured to emit light having a predetermined wavelength, and a light receiving element configured to receive returned light from a subject with respect to the emitted light. The device also includes an attachment determination unit configured to determine whether the device is attached to the subject with respect to the light receiving element based on an amount of returned light received, and a measurement-point determination unit configured to determine whether or not a point where the device is attached to the subject is suitable for measuring the biological information based on the amount of returned light received, when the attachment determination unit determines that the device is,attached to the subject.

### BRIEF DESCRIPTION OF THE DRAWINGS

Other objects and further features of embodiments will become apparent from the following detailed description when read in conjunction with the accompanying drawings, in which:

FIGS. 1A and 1B are perspective views illustrating an example of a schematic configuration of a device for measuring biological information according to an embodiment;

FIG. 2 is a plan view illustrating an example of arrangement of a first light emitting unit, a second light emitting unit, and a light receiving unit in the device for measuring biological information according to the embodiment;

FIG. 3 is a cross-sectional view along line A-A′ of FIG. 1;

FIG. 4 is a block diagram illustrating an example of a configuration of a sensor module according to the embodiment.

FIG. 5 is a flowchart illustrating an example of a process for determination of the attachment to a subject and determination of a point for measuring the biological information according to the embodiment; and

FIG. 6 is a plan view illustrating an example of arrangement of a first light emitting unit, a second light emitting unit, and a light receiving unit according to modifications of the embodiment.

### DESCRIPTION OF THE PREFERRED EMBODIMENTS

With reference to the drawings, explanation will be provided hereinafter for a device for measuring biological information. In each figure, an X-Y-Z coordinate is indicated as a reference coordinate, and an X-Y plane is a plane perpendicular to a Z1-Z2 direction. In the following description, a Z1 direction may be referred to as upward, a Z2 direction may be referred to as downward, and a view seen along the Z1-Z2 direction may be referred to as a plan view. Note that in the following description, the same reference numerals are used to denote same elements; accordingly, for the elements described once, the description may be omitted as appropriate.

(Configuration of Device for Measuring Biological Information)

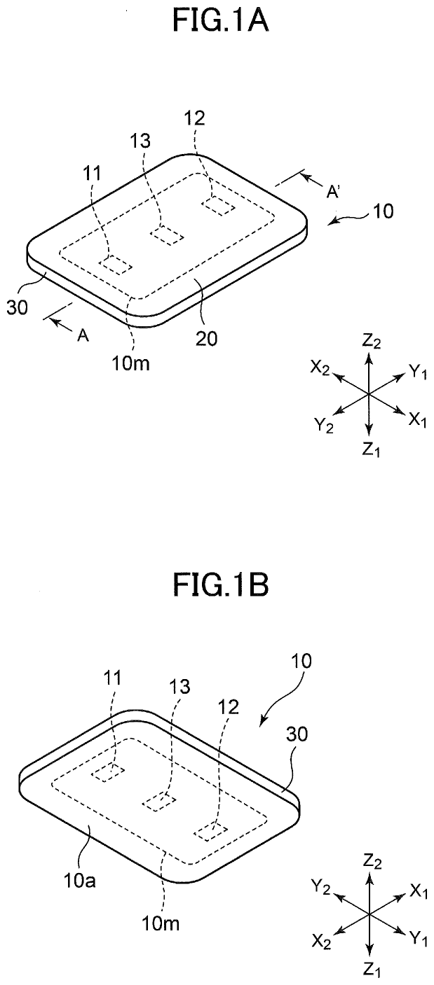

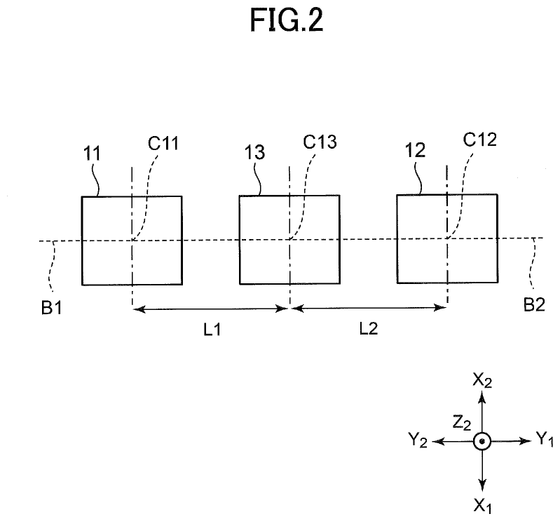

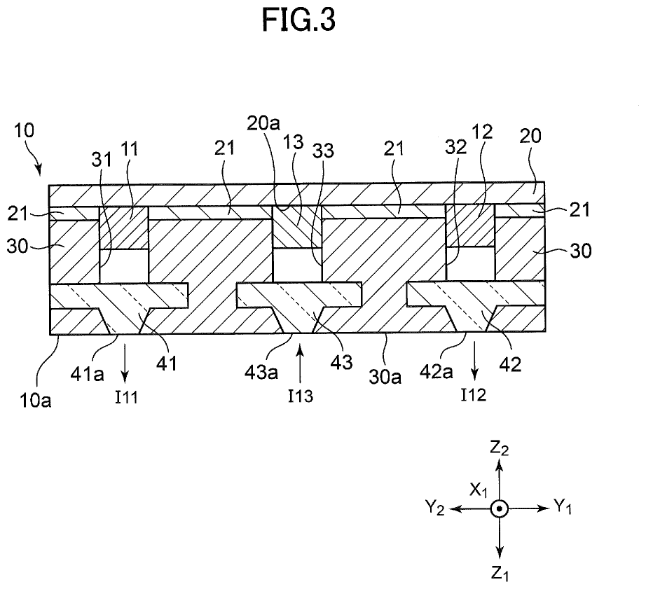

FIGS. 1A and 1B are perspective views illustrating an example of a schematic configuration of a device 10 for measuring biological information according to an embodiment. FIG. 1A is a perspective view when viewed from a side of a substrate 20, and FIG. 1B is a perspective view when viewed from a side of a light receiving and emitting surface 10a, which is disposed on an opposite side of the substrate 20. FIG. 2 is a plan view illustrating an example of arrangement of a first light emitting unit 11, a second light emitting unit 12, and a light receiving unit 13 in the device 10 for measuring biological information according to the embodiment. FIG. 3 is a cross-sectional view along line A-A′ of FIG. 1.

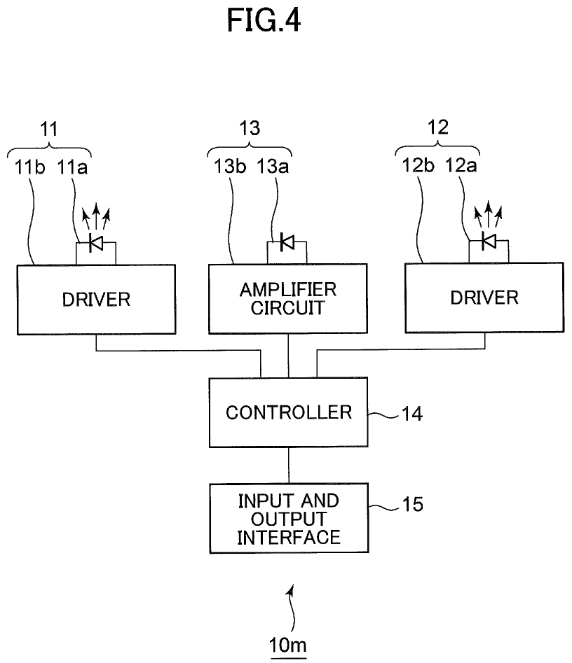

The device 10 for measuring biological information (which may be referred to as a device 10 hereafter) is attached to a subject, e.g., the skin (point) of a human body, by close contact. The device 10 is a device that measures information on substances from blood, which is used as biological information. The device 10 includes a sensor module 10m as illustrated in FIG. 4. The sensor module 10m includes two light emitting units 11 and 12 and a light receiving unit 13, which are disposed on an upper surface 20a (FIG. 3) (a surface toward the Z1 direction) of the substrate 20.

As illustrated in FIG. 3, the light emitting unit 11 of the two light emitting units 11 and 12 emits light (which may be hereafter referred to as measurement light) I11 having a predetermined wavelength, and radiates (emits) the light onto the subject, as measurement light. The light emitting unit 12 emits light (which may be hereafter referred to as measurement light) I12 having a predetermined wavelength, and radiates (emits) the light onto the subject, as the measurement light. The light receiving unit 13 receives returned light I13 from the subject with respect to the measurement light emitted by each of the light emitting units 11 and 12. In the present embodiment, the returned light from the subject includes light propagating inside the subject, e.g., in blood vessels, light diffused inside the subject, and light reflected or diffused on a given surface. Each of the light I11 and the light I12 is radiated on a light receiving and emitting surface 10a, which is disposed opposite to the substrate 20 in the Z1-Z2 direction. Also, the returned light I13 is received on the light receiving and emitting surface 10a. The device 10 is attached such that the light receiving and emitting surface 10a contacts closely with the subject. Note that the sensor module 10m including the two light emitting units 11 and 12 and the light receiving unit 13 will be described in detail below.

As illustrated in FIG. 2, the first light emitting unit 11, the light receiving unit 13, and the second light emitting unit 12 are arranged in this order from a Y2 side to a Y1 side along the Y1-Y2 direction. A center-to-center distance between the center C11 of a plane represented as the first light emitting unit 11 and the center C13 of a plane represented as the light receiving unit 13 is set as a first distance L1. A center-to-center distance between the center C12 of a plane represented as the second light emitting unit 12 and the center C13 of the plane represented as the light receiving unit 13 is set as a second distance L2. The first distance L1 and the second distance L2 may have a same distance. Each of the first distance L1 and the second distance L2 may be set by Equation (1).

0.7≤L2/L1≤1.3 (1)

Further, each of the distances L1 and L2 may be in a range of 4 mm or more as well as 11 mm or less.

When each of the distances L1 and L2 satisfies Equation (1) above and/or the above range, variations in how deeply the measurement light radiated by each of the two light emitting elements 11a and 12a reaches in regions of the subject can be decreased within certain limits. Thereby, variations in the measurement of biological information performed based on the measurement light from each of the light emitting elements 11a and 12a are able to be decreased.

As illustrated in FIGS. 1 and 3, the device 10 includes a housing 30. The housing 30 is provided on the upper surface 20a of the substrate 20 through an adhesive layer 21. Inside the housing 30, the following is formed: an opening 31 for first emission, which is formed in an emission passageway of the light I11 from the first light emitting unit 11, an opening 32 for second emission, which is formed in an emission passageway of the light I12 from the second light emitting unit 12, and an opening 33 for light received, which is formed in a light received passageway of the returned light I13 received by the light receiving unit 13. The first light emitting unit 11 is disposed in the opening 31 for first emission. The second light emitting unit 12 is disposed in the opening 32 for second emission. The light receiving unit 13 is disposed in the opening 33 for light received. Outgoing light from the first emitting unit 11 is propagated in the opening 31 for first emission, and outgoing light from the second emitting unit 12 is propagated in the opening 32 for second emission.

The housing 30 is formed of material capable of blocking out light, such as metal or resin. When the housing 30 is formed of material capable of blocking out light, outgoing light from each of the first light emitting unit 11 and the second light emitting unit 12 can be prevented from entering the light receiving unit 13 directly, without being propagated inside the subject. For this reason, information necessary to measure biological information can be easily obtained accurately, thereby enabling measurement with precise accuracy. For example, when the housing 30 is formed of metallic material, the housing 30 can serve as a heat dissipating member that allows heat caused by the two light emitting units 11 and 12 to be dissipated to the external air. Alternatively, when the housing 30 is formed of resin material, its elasticity allows the housing 30 to be disposed so as to match the shape of the skin of the subject. Accordingly, adhesion can be improved.

In the device 10, three transmissive members 41, 42 and 43 are disposed so as to cover respective upper areas of the opening 31 for first emission, the opening 32 for second emission, and the opening 33 for light received. The light radiated by the first light emitting unit 11 is, as measurement light, propagated from the opening 31 for first emission to the transmissive member 41, and then is propagated forward to the outside of an upper side of the device 10. The light irradiated by the second light emitting unit 12 is, as measurement light, propagated from the opening 32 for second emission to the transmissive member 42, and then is propagated forward to the outside of an upper side of the device 10. Returned light from the subject with respect to the above measurement light is propagated through the transmissive member 43, and then is propagated forward in the opening 33 for light received. As a result, the returned light is received by the light receiving unit 13. For example, each of the transmissive members 41, 42 and 43 is formed of PET (polyethylene terephthalate). These three transmissive members 41, 42 and 43 are secured to the housing 30 with adhesives, and their upper end surfaces 41a, 42a and 43a form a same surface that constitutes the upper surface 30a of the housing 30, which serves as the light receiving and emitting surface 10a. In such a manner, the housing 30 and the transmissive members 41, 42 and 43 are adhered all together to the subject.

(Configuration of Sensor Module)

FIG. 4 is a block diagram illustrating an example of a configuration of the sensor module 10m.

The sensor module 10m includes a pair of light emitting units 11 and 12, a light receiving unit 13, a controller 14, and an input and output interface 15.

As illustrated in FIG. 4, the first light emitting unit 11 includes a first light emitting element 11a, and the second light emitting unit 12 includes a second light emitting element 12a. Each of the first light emitting element 11a and the second light emitting element 12a emits measurement light including near-infrared light with an emission wavelength of 600 nm or more as well as 804 nm or less. As an example, such an emission wavelength may be 758 nm or more with 762 nm or less. Each of the first light emitting element 11a and the second light emitting element 12a is a light-emitting diode element or a laser element.

Note that each of the first light emitting unit 11 and the second light emitting unit 12 may further include a light emitting element that emits measurement light including near-infrared light of 806 nm or more as well as 995 nm or less, which is different from an emission wavelength of light emitted by each of the first light emitting element 11a and the second light emitting element 12a. Thereby, the measurement used in such a manner can indicate biological information different from the biological information obtained by the two light emitting elements 11a and 12a irradiating a subject with measurement light.

The light receiving unit 13 includes a light receiving element 13a that converts near-infrared light, which is radiated by the first light emitting unit 11 or the second light emitting unit 12 and that is used as returned light propagated inside a body of the subject, e.g., returned light reflected by blood that circulates along blood vessels, into an electrical signal. A photodiode is an example of the light receiving element 13a. A DC current with a level corresponding to an amount of light received flows to the light receiving element 13a. The light receiving element 13a outputs, as a received light signal, an electrical signal in accordance with the level of the DC current (which may be hereinafter referred to as a DC level).

The two light emitting units 11 and 12 and the light receiving unit 13 may be integrally configured as a light receiving and emitting unit. Alternatively, the sensor module 10m may be a package that contains the two light emitting units 11 and 12, the light receiving unit 13, the controller 14, and the input and output interface 15.

The first light emitting unit 11 includes a driver 11b that drives the first light emitting element 11a, and the second light emitting unit 12 includes a driver 12b that drives the second light emitting element 12a. The light receiving unit 13 includes an amplifier circuit 13b that amplifies received light signals outputted by the light receiving element 13a. These components 11b, 12b and 13b may be integrated as one chip.

The controller 14 includes a microcomputer. The controller 14, which serves as a light emitting controller, transmits a timing signal to each of the driver 11b of the first light emitting unit 11 and the driver 12b of the second light emitting unit 12. Accordingly, the controller 14 controls the first light emitting unit 11 and the second light emitting unit 12 such that they each emit near-infrared light at a predetermined timing. Specifically, in measurement of biological information, the controller 14 causes the first light emitting unit 11 and the second light emitting unit 12 to simultaneously emit light. In determination of the attachment to a subject, the controller 14 causes the first light emitting unit 11 and the second light emitting unit 12 to sequentially emit light at predetermined intervals. In determination of a point for measuring the biological information, the controller 14 causes the first light emitting unit 11 and the second light emitting unit 12 to alternately emit light at predetermined intervals. In the present embodiment, light emission of measurement of biological information and determination of the attachment to a subject, as well as determination of a point for measuring the biological information, are each achieved at a different timing. Thereby, the determination of the attachment to a subject, as well as the determination of a point for measuring the biological information, can be reliably made. Accordingly, the measurement or output of biological information can be avoided in a case where the device 10 is not attached to a suitable point.

With use of a built-in analog-to-digital conversion circuit, the controller 14, which serves as a biological information measuring unit, converts an amplified received light signal, which is outputted by the amplifier circuit 13b of the light receiving unit 13, into signal information in a digital format capable of being processed. The controller 14 estimates information (biological information) related with blood circulating along blood vessels inside a subject, based on the converted signal information. As the biological information estimated by the controller 14, a change in hemoglobin in the blood (an amount of changes in Hb), a change in oxygen ratios in the blood (oxygen saturation), or the like is used in measurement through the use of returned light from a subject with respect to near-infrared light radiated by each of the first light emitting element 11a and the second light emitting element 12a.

Oxygenated hemoglobin and deoxygenated hemoglobin are equal in absorbance at a wavelength of 805 nm. At wavelengths of more than 805 nm, the absorbance of oxygenated hemoglobin is greater than that of deoxygenated hemoglobin. At wavelengths of less than 805 nm, the absorbance of oxygenated hemoglobin is smaller than that of deoxygenated hemoglobin. Accordingly, when a human body as a subject is irradiated with near infrared light having a wavelength of 804 nm or less by each of the first light emitting element 11a and the second light emitting element 12a, the absorbance of deoxygenated hemoglobin can be preferentially measured. Deoxygenated hemoglobin tends to have less change in absorbance with respect to time than oxygenated hemoglobin, and thus a pulsation or/and a plethysmogram of the subject can be measured more accurately.

Further, the sensor module 10m can perform measurement at a sampling rate of about 10 milliseconds, and thus information related with blood can be obtained continuously.

Note that, when each of the first light emitting unit 11 and the second light emitting unit 12 further includes a light emitting element that emits measurement light including near-infrared light, of which the emission wavelength is 806 nm or more as well as 995 nm or less, information obtained from blood circulating along blood vessels inside a subject, such as a pulsation of blood, a blood flow rate, or a flow velocity, etc., can be acquired. Further, a change in oxygen ratios in the blood (oxygen saturation) or information related with such a change can be acquired based on a result of the measurement by light including near-infrared light of 804 nm or less, as well as a result of the measurement by light including near-infrared light of 806 nm or more with 995 nm or less, where such light is radiated by each of the two light emitting elements 11a and 12a.

Further, the controller 14, which serves as an attachment determination unit, determines whether the device 10 is suitably attached to a subject based on a DC level corresponding to an amount of returned light received by the light receiving unit 13. The returned light is received every time the first light emitting element 11a and the second light emitting element 12a each emit measurement light at predetermined time intervals. The controller 14 determines that the device 10 is suitably attached to a subject, when a DC level of the amount of returned light received with respect to each of the first light emitting unit 11 and the second light emitting unit 12 is equal to or greater than a threshold value V1. As an example, the threshold value V1 may be a smallest value of DC level capable of measuring the biological information. In the present embodiment, being suitably attached means that the device 10 is attached to a human body as a subject in such a manner that returned light capable of performing measurement of biological information is able to be received. By way of example, the upper surface 30a of the housing 30 has an adhesive, and the device 10 is attached to a human body with the adhesive. In such a manner, in determination of the attachment to a subject, the controller 14 can determine whether such attachment with the adhesive is suitable for measuring the biological information.

Note that a predetermined value without reference to suitability of measuring the biological information may be used as the threshold value V1. In this case, it is determined that the device 10 is suitably attached upon detecting an amount of returned light received being equal to or greater than a certain value.

In contrast, with respect to either or both of the first light emitting unit 11 and the second light emitting unit 12, when a DC level of an amount of returned light received is smaller than a threshold value V1, the controller 14 determines that the device 10 is not suitably attached to a subject. In such a manner, the threshold value V1 can be used to determine whether the device 10 is suitably attached, thereby determining a state of attachment objectively and accurately.

Note that, in determination of the attachment to a subject, one of the first light emitting unit 11 and the second light emitting unit 12 may be lighted only. In such a manner as well, the controller 14 determines whether the device 10 is suitably attached to a subject based on a DC level that corresponds to an amount of returned light received being equal to or greater than the threshold value V1.

Alternatively, the first light emitting unit 11 and the second light emitting unit 12 can be lighted simultaneously to make determination of the attachment to a subject. In such a manner, the controller 14 determines whether the device 10 is suitably attached to a subject based on a DC level that corresponds to an amount of returned light received being equal to or greater than a threshold value V2. As an example, the threshold value V2 is a double value of the threshold value V1 for used in determination by the first light emitting unit 11 and the second light emitting unit 12 alternately lighting.

The controller 14, which serves as a measurement-point determination unit, determines whether a point where the device 10 is attached to a subject is suitable for measuring the biological information based on a DC level corresponding to an amount of returned light received by the light receiving unit 13. The returned light refers to returned lights corresponding to respective measurement lights, emitted at respective timings by the first light emitting element 11a and the second light emitting element 12a for a predetermined time-interval. The controller 14 calculates a total of amounts of returned light received with respect to measurement light from the first light emitting element 11a, as well as measurement light emitted by the second light emitting element 12a at a timing subsequent to the emission of the first light emitting element 11a. When such a total is equal to or greater than a predetermined value V3, the controller 14 determines that the point is suitable for measuring the biological information, and then marks the point as a sweet spot.

Note that the predetermined value V3 may be greater than the threshold value V1 used in determination of the attachment to a subject. Thereby, a range of amounts of returned light received for use in determination of a point for measuring the biological information can be decreased. For this reason, a throughput of a computation process is able to be decreased, thereby allowing high-speed processing.

For example, when determining that the point is suitable for measuring the biological information, the controller 14 causes a display unit, which is not illustrated in the figures, to display information for indicating that the point is suitable. In contrast, when the above total of amounts is smaller than the predetermined value V3, the controller 14 determines that the point is not suitable for measuring the biological information. In this case, the controller 14 causes an alert unit, which is not illustrated in the figures, to sound beeps, by way of example.

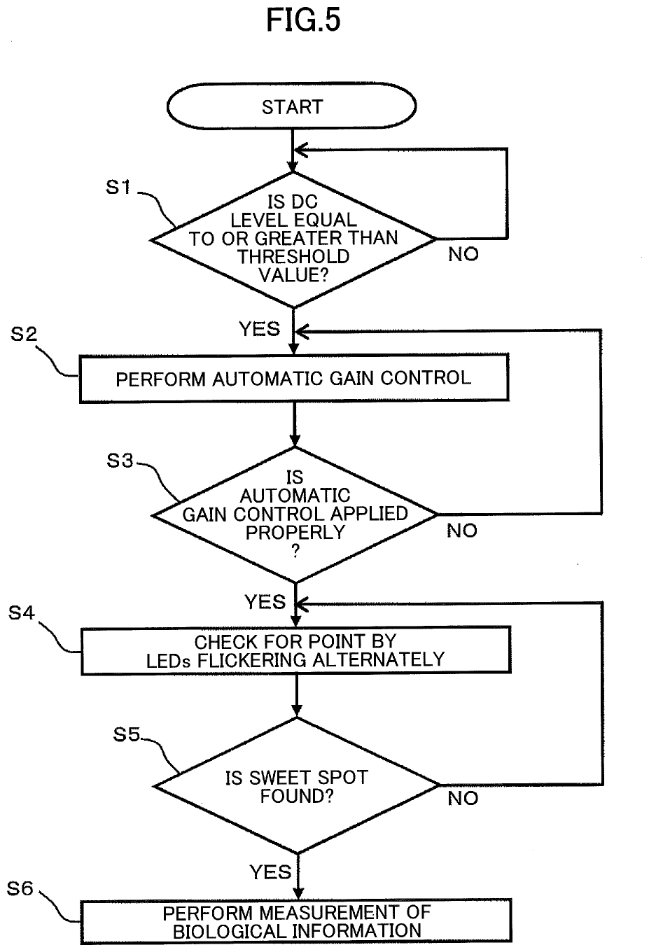

Hereafter, with reference to FIG. 5, a process for determination of the attachment to a subject as well as determination of a point for measuring the biological information is described by way of example. FIG. 5 is a flowchart illustrating a process for determination of the attachment to a subject as well as determination of a point for measuring the biological information.

First, the device 10 is attached to the skin of a human body (target) as a subject, and then the first light emitting element 11a of the first light emitting unit 11 and the second light emitting element 12a of the second light emitting unit 12 are lighted sequentially under control of the controller 14. The interval of the lighting is 0.01 seconds, for example. In such a manner, the human body is sequentially irradiated with near-infrared light that is used as measurement light from each of the first light emitting element 11a and the second light emitting element 12a. Subsequently, returned light from the human body with respect to the near-infrared light is received by the light receiving element 13a of the light receiving unit 13. A received light signal outputted by the light receiving element 13a is amplified by the amplifier circuit 13b, and then is transmitted to the controller 14. The controller 14 determines whether or not a DC level corresponding to the received light signal transmitted by the amplifier circuit 13b is equal to or greater than the threshold value V1 (step S1).

In determination in step S1, with respect to each of the first light emitting unit 11 and the second light emitting unit 12, when a DC level of an amount of returned light received is equal to or greater than the threshold value V1 (YES in step S1), the controller 14 determines that the device 10 is suitably attached to the subject, and then performs automatic gain control (step S2). On the other hand, with respect to either or both of the first light emitting unit 11 and the second light emitting unit 12, when a DC level is smaller than the threshold value V1 (NO in step S1), the controller 14 determines that the device 10 is not suitably attached to the subject. In such a manner, the controller 14 causes a display unit that is not illustrated in the figures to display a message or the like for requesting to attach to a subject again. After reattachment, the controller 14 causes the first light emitting element 11a and the second light emitting element 12a to light sequentially, and again makes determination of the attachment to a subject.

In automatic gain control in step S2, the controller 14 transmits an instruction signal to each of the driver 11b and 12b, so as to increase a drive current flowing to each of the first light emitting element 11a and the second light emitting element 12a. Further, the controller 14 monitors an amount of light received at a predetermined interval by the light receiving element 13a, and determines whether or not a DC level corresponding to an amount of light received has reached a target value (target level) that is preset and stored in a built-in memory (step S3). When a DC level reaches the target value, the controller 14 determines that automatic gain control is applied properly (YES in step S3). While a DC level does not reach the target value (NO in step S2), auto gain control continues to be performed. As an example, the target value is set in consideration of a region to be measured, or/and biological information of a target as a subject (e.g., body weight, height, body fat rate, age, sex).

After the automatic gain control is applied properly and a DC level reaches the target value, in determination of a point for measuring the biological information, the controller 14 causes the first light emitting element 11a and the second light emitting element 12a to light alternately at predetermined intervals, and checks for a point where the device 10 is attached to the subject, based on an amount of returned light received, in order to find a sweet spot (step S4).

In checking for a point, the controller 14 calculates the total of amount of returned light received with respect to measurement light from the first light emitting element 11a, as well as measurement light emitted by the second light emitting element 12a at a timing subsequent to the emission of the first light emitting element 11a. When the total of amount of returned light received is equal to or greater than the predetermined value V3, the controller 14 determines that the point is suitable for measuring the biological information. As a result, such a point is marked as a sweet spot (YES in step S5). In contrast, when a total of amounts of returned light received is smaller than the predetermined value V3 (NO in step S5), the controller 14 sends a notification to a subject with beeps or the like. Accordingly, position or orientation with respect to the device 10 can be changed by the subject, and then the controller 14 continues to check for a changed point in order to find a sweet spot.

After the sweet spot is found (YES in step S5), the controller 14 performs measurement of biological information for such a sweet spot (step S6).

In such a configuration described above, according to the above embodiments, it is possible to determine whether or not the device 10 is attached to a point suitable for measuring the biological information, thereby allowing measurement at an optimum point. Accordingly, accurate measurement can be performed as desired. Further, after it is determined that the device 10 is suitably attached, determination of a point for measuring the biological information is made. Thereby, a range of amounts of returned light received for use in determination of a point for measuring the biological information can be decreased. For this reason, a throughput of a computation process is able to be decreased, thereby allowing high-speed processing. Furthermore, the two light emitting elements 11a and 12a alternately emit light so that a subject can be irradiated with the light at a high speed. Thereby, determination of a point for measuring the biological information can be made quickly with precise accuracy. In the present embodiment, the two light emitting elements 11a and 12a are used to make determination of the attachment to a subject with determination of a point for measuring, as well as performing measurement of biological information. Thereby, it is possible to decrease the size of the device as well as reducing the cost of components.

Modifications of the embodiments are described hereafter.

In the embodiments, it is determined that the point is suitable for measuring the biological information when a total of amounts of light received with respect to the first light emitting element 11a and the second light emitting element 12a is equal to or greater than a predetermined value. However, instead of the above, when a difference between amounts of light received with respect to the first light emitting element 11a and the second light emitting element 12a is equal to or smaller than a predetermined value, and further, each of the amounts of light received is equal to or greater than a predetermined value, it may be determined that such a point is suitable for measuring the biological information.

Accordingly, the following case is able to be identified: amounts of light received with respect to respective light emitting elements are all increased, and further, variations in the amounts of light received are decreased. In such a case, a point for measuring the biological information is able to be marked as an optimum point.

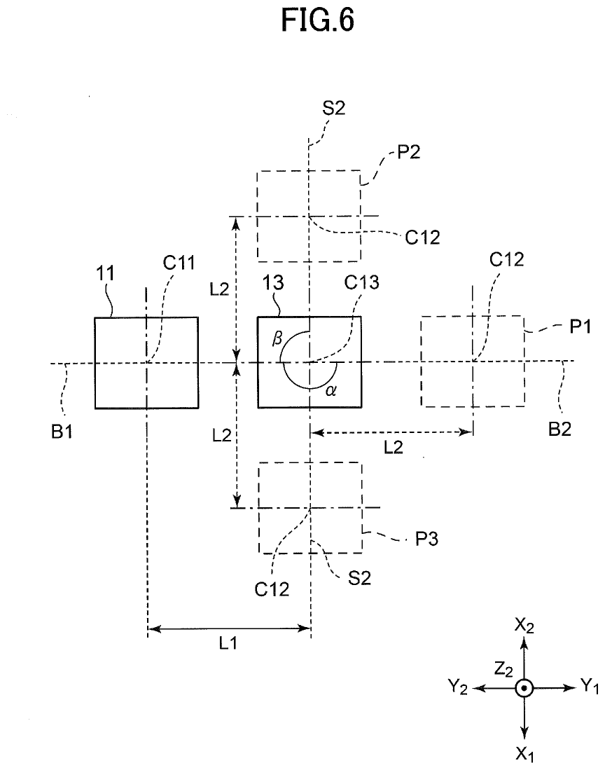

FIG. 6 is a plan view illustrating an example of arrangement of a first light emitting unit 11, a second light emitting unit 12, and a light receiving unit 13 according to the modification. In the above embodiments, as illustrated in FIG. 2, the first light emitting unit 11, the light receiving unit 13, and the second light emitting unit 12 have been arranged in this order in a straight line along the Y1-Y2 direction. In other words, with reference to FIG. 6, the following has been described: the first light emitting unit 11 and the light receiving unit 13 are disposed along the Y1-Y2 direction, and further, the second light emitting unit 12 is disposed at a position P1. An angle α between a straight line B1, which connects the center C11 of a plane represented by the first light emitting unit 11 and the center C13 of a plane represented by the light receiving unit 13, and a straight line B2, which connects the center C13 of a plane represented by the light receiving unit 13 and the center C12 of a plane represented by the second light emitting unit 12, is 180 degrees. However, the position of the second light emitting unit 12 may be changed such that the angle a with respect to the first light emitting unit 11, the light receiving unit 13, and the second light emitting unit 12 may be set in a range of 90 degrees or more to 180 degrees or less. For example, as illustrated at a position P2 or a position P3 in FIG. 6, the second light emitting unit 12 may be disposed such that an angle β between a straight line B1 and a straight line B2 is 90 degrees. A center-to-center distance between the second light emitting unit 12 and the light receiving unit 13 may be L2 in the case of any of positions P1, P2 and P3. Thereby, light emitting element(s) can be flexibly arranged in accordance with the shape or the like of a given region of a subject, in terms of a size, a degree of curvature, an amount of muscle or fat, or/and thickness of the blood vessel, etc., for example. Also, the attachment to a subject can be easily adjusted, thereby allowing accurate measurement of biological information.

In the above embodiments and modifications, by way of example, two light emitting units are provided, but the number of light emitting units may be three or more.

In the above embodiments and modifications, the transmissive members 41, 42 and 43 and the upper surface 30a of the housing 30 constitute a same surface (light receiving and emitting surface 10a). However, upper ends of the transmissive members 41, 42 and 43 may be configured so as to be protruded upwardly (in the Z1 direction) from the upper surface 30a of the housing 30. In such a configuration, by pressing the device 10 to the skin, adhesion between each of the transmissive members 41, 42 and 43 and a subject can be secured.

Alternatively, the upper surface 30a of the housing 30 can be configured so as to be disposed above respective upper ends of the transmissive members 41, 42 and 43. In such a configuration, by pressing the device 10 to the skin to adhere the skin to the housing 30, a distance between the skin and each of the transmissive members 41, 42 and 43 can be maintained at an approximately constant level, as desired.

As described above, the embodiments and modifications of the present disclosure have been described in detail, but are not limited to the examples described above. It will be appreciated by those skilled in the art that various modifications or changes to the foregoing embodiments are made within the scope of the present invention or the equivalent thereof.

### INDUSTRIAL APPLICABILITY

As described above, a device for measuring biological information according to the present disclosure is useful in that it is possible to determine whether or not the device is attached at a point suitable for measuring the biological information.

Embodiments of the present disclosure provide a device for measuring biological information whereby it is possible to determine whether or not the device is attached to a point suitable for measuring the biological information, thereby allowing for measurement at an optimum point. Accordingly, accurate measurement can be achieved as desired.

In a first manner, a device for measuring biological information includes at least one light emitting element configured to emit light having a predetermined wavelength, a light receiving element configured to receive returned light from a subject with respect to the emitted light, an attachment determination unit configured to determine whether the device is suitably attached to the subject with respect to the light receiving element based on an amount of returned light received, and a measurement-point determination unit configured to determine whether or not a point where the device is attached to the subject is suitable for measuring the biological information based on the amount of returned light received, when the attachment determination unit determines that the device is suitably attached to the subject.

In such a manner, it is possible to determine whether or not the device is attached to a point suitable for measuring the biological information, thereby allowing for measurement at an optimum point. Accordingly, accurate measurement can be achieved as desired. Further, determination of a point for measuring the biological information is achieved after it is determined that the device is suitably attached, thereby decreasing a range of amounts of light received for use in determination of a point for measuring the biological information. Accordingly, the throughput of such a determination process is decreased, and thus high-speed processing can be achieved.

In a second manner, according to the first manner, the attachment determination unit may be configured to determine that the device is suitably attached to the subject upon detecting the amount of returned light received being equal to or greater than a predetermined value.

In such a manner, an amount of returned light received can meet or exceed a suitable level, thereby decreasing the throughput for use in determination of a point for measuring the biological information accordingly.

In a third manner, according to the first manner, the at least one light emitting element is a plurality of light emitting elements, and the measurement-point determination unit may be configured to determine whether or not the point is suitable based on amounts of returned light received with respect to the light that the respective light emitting elements alternately emit.

In such a manner, a point suitable for measuring the biological information is identified, thereby allowing accurate measurement as desired.

In a fourth manner, according to the third manner, the measurement-point determination unit may be configured to determine that the point is suitable upon detecting a total of amounts of returned light received with respect to the light that the respective light emitting elements alternately emit, the total of amounts of returned light received being equal to or greater than a predetermined value.

In such a manner, a case where amounts of returned light received with respect to respective light emitting elements are all increased can be identified, thereby marking an optimum point accordingly.

In a fifth manner, according to the third manner, the measurement-point determination unit may be configured to determine that the point is suitable upon detecting a difference between the amounts of returned light received with respect to the light that the respective light emitting elements alternately emit, as well as detecting each of the amounts of returned light received being equal to or greater than a predetermined value, the difference being equal to or smaller than a predetermined value.

In such a manner, the following case can be identified: amounts of returned light received with respect to respective light emitting elements are all increased, and further, variations in the amounts of returned light received are decreased. Thereby, a point for measuring the biological information can be marked as an optimum point accordingly.

In a sixth manner, according to any one of the third manner to the fifth manner, the plurality of light emitting elements may be each configured to emit the light having a same wavelength.

In such a manner, a process of processing received light signals, which are obtained when the plurality of light emitting elements alternately emit light in determination of a point for measuring the biological information, can be easily executed.

In a seventh manner, according to any one of the third manner to the sixth manner, when a distance between the light receiving element and one light emitting element of the plurality of light emitting elements is set as L1, a distance L2 between each of the remainder of the light emitting elements and the light receiving element may be defined by 0.7≤L2/L1≤1.3.

In an eighth manner, according to the seventh manner, each of the distance L1 and the distance L2 may be 4 mm or more with 11 mm or less.

In such a manner, variations in how deeply the light radiated by each of the two light emitting elements 11a and 12a reaches in regions of the subject can be decreased within certain limits. Thereby, variations in the measurement of biological information, which are performed based on the light from each of the light emitting elements, can be decreased.

In a ninth manner, according to any one of the third manner to the eighth manner, the at least one light emitting element is two light emitting elements being a first light emitting element and a second light emitting element, and an angle between a first line connecting the first light emitting element and the light receiving element and a second line connecting the light receiving element and the second light emitting element may be 90 degrees or more with 180 degrees or less.

In such a manner, light emitting element(s) can be flexibly arranged in accordance with the shape or the like of a given region of a subject. Accordingly, the attachment to a subject can be easily adjusted, thereby allowing accurate measurement of biological information.

According to the manners described above, it is possible to determine whether or not the device is attached to a point suitable for measuring the biological information, thereby allowing for measurement at an optimum point. Accordingly, accurate measurement can be achieved as desired.

What is claimed is:

1. A device for measuring biological information comprising;
at least one light emitting element configured to emit light having a predetermined wavelength;
a light receiving element configured to receive returned light from a subject with respect to the emitted light;
an attachment determination unit configured to determine whether the device is attached to the subject with respect to the light receiving element based on an amount of returned light received; and
a measurement-point determination unit configured to determine whether or not a point where the device is attached to the subject is suitable for measuring the biological information based on the amount of returned light received, when the attachment determination unit determines that the device is attached to the subject.

2. The device according to claim 1, wherein the attachment determination unit is configured to determine that the device is attached to the subject upon detecting the amount of returned light received being equal to or greater than a predetermined value.

3. The device according to claim 1, wherein the at least one light emitting element is a plurality of light emitting elements, and
wherein the measurement-point determination unit is configured to determine whether or not the point is suitable based on amounts of returned light received with respect to the light that the respective light emitting elements alternately emit.

4. The device according to claim 3, wherein the measurement-point determination unit is configured to determine that the point is suitable upon detecting a total of amounts of returned light received with respect to the light that the respective light emitting elements alternately emit, the total of amounts of returned light received being equal to or greater than a predetermined value.

5. The device according to claim 3, wherein the measurement-point determination unit is configured to determine that the point is suitable upon detecting a difference between the amounts of returned light received with respect to the light that the respective light emitting elements alternately emit, as well as detecting each of the amounts of returned light received being equal to or greater than a predetermined value, the difference being equal to or smaller than a predetermined value.

6. The device according to claim 3, wherein each light emitting element among the plurality of light emitting elements is configured to emit light having a same wavelength.

7. The device according to claim 3, wherein when a distance between the light receiving element and one light emitting element among the plurality of light emitting elements is set as L1, a distance L2 between each of a remainder of the light emitting elements and the light receiving element is defined by:
0.7≤L2/L1≤1.3.

8. The device according to claim 7, wherein each of the distance L1 and the distance L2 is 4 mm or more and is 11 mm or less.

9. The device according to claim 3, wherein the at least one light emitting element is two light emitting elements being a first light emitting element and a second light emitting element, and
wherein an angle between a first line connecting the first light emitting element and the light receiving element and a second line connecting the light receiving element and the second light emitting element is 90 degrees or more and is 180 degrees or less.
